(19) United States
(12) Patent Application Publication
(10) Pub. No.: US 2009/0099573 A1
(43) Pub. Date: Apr. 16, 2009

(54) Apparatus and Method for Treating Eustachian Tube Dysfunction

(76) Inventor: Donald Gonzales, San Antonio, TX (US)

Correspondence Address: FULBRIGHT & JAWORSKI L.L.P., 600 CONGRESS AVE., SUITE 2400, AUSTIN, TX, 78701, US

(21) Appl. No.: 12/247,825

(22) Filed: Oct. 8, 2008

Related U.S. Application Data
(60) Provisional application No. 60/978,873, filed on Oct. 10, 2007.

(51) Int. Cl. A61F 11/00

(52) U.S. Cl. 606/108

(57) ABSTRACT

A method and apparatus for treating Eustachian tube dysfunction. The apparatus may include a tube with an aperture extending through the tube and a retention mechanism for retaining the tube in the Eustachian tube. The method may include endoscopically inserting the tube through the nasopharynx and into the Eustachian tube.

### CROSS-REFERENCE TO RELATED APPLICATIONS

This application claims priority to U.S. Provisional Patent Application Ser. No. 60/978,873, filed Oct. 10, 2007, the entire contents of which are expressly incorporated herein by reference.

### FIELD OF THE INVENTION

The present disclosure relates generally to apparatus and methods for treating Eustachian tube dysfunction. The present disclosure relates more specifically to apparatus and methods to reduce blockage of a Eustachian tube.

### BACKGROUND INFORMATION

Eustachian tube dysfunction (ETD) is a common problem for both children and adults. The Eustachian tube (or auditory tube) acts as a pressure equalizing tube that extends from the lateral nasopharynx to the middle ear. The tube is cartilaginous from the nasopharynx to the isthmus, at which point it is bony for the rest of the path to the middle ear. When the Eustachian tube is obstructed either through anatomical or inflammatory reasons, the middle ear is not able to equalize pressure which can lead to negative pressure and fluid build-up or retraction of the tympanic membrane.

ETD can lead to many otologic problems such as chronic otitis media, retraction of the tympanic membrane, hearing loss and cholesteatoma. To date, a typical treatment for this problem involves medical therapy such as antihistamines and decongestants. A typical surgical therapy includes the placement of pressure equalization (PE) tubes in the tympanic membrane. However, PE tube placement does not treat the underlying problem, and only helps prevent complications from prolonged ETD. PE tube placement is not a benign procedure and can lead to many adverse consequences, especially for adults with ETD. The placement of the PE tube requires an incision in the tympanic membrane with the tube, creating a non-natural drainage and equalization of pressure in the middle ear. Complications can include damage to chronic otorrhea, retained tube, perforation of the tympanic membrane and damage to surrounding structures. In the pediatric population, ETD is caused more commonly by the anatomy of the Eustachian tube, which generally resolves with age. In the adult, the causes can be multifactorial but are mainly due to inflammation of the mucosa of the opening of the Eustachian tube in the nasopharynx. The inflammation can be caused by many factors, such as gastroesophageal reflux disease, allergic reactions, viral or bacterial infection, and smoking. Because ETD in the adult is not self-limited, most adults require multiple sets of PE tubes.

Given the lack of adequate surgical or medical treatment for chronic Eustachian tube dysfunction, there remains a need in the art for an effective apparatus and method for treatment. The desired treatment should reduce the need and complications of repeated PE tube placements.

### SUMMARY

Exemplary embodiments of the present disclosure comprise a method and apparatus of treating Eustachian tube dysfunction (ETD). In certain embodiments, a tube can be guided through the nasopharynx and inserted into the opening of the Eustachian tube in the nasopharnyx to allow a passageway through the Eustachian tube that would otherwise be obstructed. Exemplary embodiments provide an apparatus and method for treatment of ETD and allowing pressure equalization for the middle ear.

Exemplary embodiments of the apparatus can allow the Eustachian tube to be unobstructed at the nasopharyngeal opening through a tube placed endoscopically. In exemplary embodiments, the apparatus may be made of a bioabsorbable or biocompatible material and may remain in place until the underlying cause of the swelling is treated successfully. In certain exemplary embodiments, the length of the apparatus is between 5 mm and 3 cm. In other exemplary embodiments, the length of the apparatus is between the following lengths: 6 mm and 29 mm; 7 mm and 28 mm; 8 mm and 27 mm; 9 mm and 26 mm; 10 mm and 25 mm; 11 mm and 24 mm; 12 mm and 23 mm; 13 mm and 22 mm; 14 mm and 21 mm; 15 mm and 20 mm; 16 mm and 19 mm; or 17 mm and 18 mm. In certain exemplary embodiments, the diameter of the apparatus is between 1 mm and 1 cm. In other exemplary embodiments, the diameter of the apparatus (either the internal aperture diameter or the outer surface diameter) is between the following distances: 2 mm and 9 mm; 3 mm and 8 mm; 4 mm and 7 mm; or 5 mm and 6 mm. In exemplary embodiments, the apparatus should be of sufficient length to extend from the opening of the eustachian tube in the nasopharnyx to the body of the cartilaginous Eustachian tube. Alternatively, the tube may extend from the opening of the Eustachian tube in the nasopharnyx to the isthmus.

Exemplary embodiments can be made of any biocompatible material. In certain embodiments, the apparatus is made of a biodegradable material. In certain embodiments, the material is a biodegradable polymer. In specific embodiments, the material is a co-polymer. In certain embodiments, the polymer is a polyester, polyanhydride, polyamide, polycarbonates, polycarbamate, polyacrylate, polymethacrylate, polystyrene, polyurea, polyether, or polyamine. In certain embodiments, the polymer is a polyester such as poly(glycolide-co-lactide) (PLGA), polyglycolic acid, poly-β-hydroxybutyrate, and polyacrylic acid ester. In certain embodiments, the apparatus or tube is made of PLGA. In certain embodiments, the polymer selected is formable and able to degrade in-vivo without producing toxic side products. Typical polymers may be selected from the family of poly-lactide, poly-glycolide, poly-caprolactone, poly-dioxanone, poly-trimethylene carbonate, and their co-polymers; however any absorbable polymer can be used. Polymers known in the art for producing biodegradable implant materials include alpha poly hydroxy acids, polyglycolide (PGA), copolymers of glycolide such as glycolide/L-lactide copolymers (PGA/PLLA), glycolide/trimethylene carbonate copolymers (PGA/TMC); polylactides (PLA), stereocopolymers of PLA such as poly-L-lactide (PLLA), Poly-DL-lactide (PDLLA), L-lactide/DL-lactide copolymers; copolymers of PLA such as lactide/tetramethylglycolide copolymers, lactide/trimethylene carbonate copolymers, lactide-valerolactone copolymers, lactide-caprolactone copolymers, polydepsipeptides, PLA/polyethylene oxide copolymers, unsymmetrically 3,6-substituted poly-1,4-dioxane-2,5-diones; polyhydroxyalkanate polymers including poly-beta-hydroxybutyrate (PHBA), PHBA/beta-hydroxyvalerate copolymers (PHBA/HVA), and poly-beta-hydroxypropionate (PHPA), poly-p-dioxanone (PDS), poly-□-valerolatone, poly-□-caprolactone, methylmethacrylate-N-vinyl pyrrolidone copolymers, polyesteramides, polyesters of oxalic acid, polydihydropyrans, polyalkyl-2-cyanoacrylates, polyurethanes (PU), polyvinyl alcohol (PVA), polypeptides, poly-beta-maleic acid (PMLA), poly(trimethylene carbonate), poly(ethylene oxide) (PEO), poly(β-hydroxyvalerate) (PHVA), poly(ortho esters), tyrosine-derived polycarbonates, and poly-beta-alkanoic acids. In certain embodiments, any absorbable polymer or combination of absorbable polymers, including co-polymers, can be used. In specific embodiments, the polymer has a molecular weight sufficient to be shaped by molding or extrusion. In certain embodiments, the polymer can be selected as is known to the art to have a desired degradation period.

In certain exemplary embodiments, the apparatus can remain in place via a retention mechanism. In certain embodiments, the retention mechanism is located proximal to one end. The retention mechanism may be one of a number of configurations. In certain exemplary embodiments, the retention mechanism may be configured as a rib, a flare, a flange, a wedge, a barb, a collar, or a hook. The retention mechanism may also be a swelling of the material from which the apparatus is constructed, so that the swelling creates a circumferential pressure and retains the apparatus in the desired location in the Eustachian tube.

In certain exemplary embodiments, the apparatus or tube may comprise a portion of a kit. The kit may comprise multiple different configurations of tubes having varying diameters or lengths. The kit may also comprise configurations having different materials of construction or different configurations for retention mechanisms. The kit may also comprise a tool or apparatus used to place the tube into the desired location within the Eustachian tube. The Eustachian tube device may be placed with the assistance of a flexible scope, either as a separate device, or acting as a sheath of the scope. The flexible scope may be passed into the opening of the Eustachian tube in the nasopharnyx to the point of retention of the tube, then the scope can be withdrawn, leaving the tube in place (similar to the Seldinger Technique).

Certain exemplary embodiments comprise a method of treating Eustachian tube dysfunction. The method may comprise: providing a tube comprising a first end, a second end, and an aperture extending through the tube; guiding the tube through a nasopharynx; inserting at least a portion of the tube into a Eustachian tube; and retaining at least a portion of the tube within the Eustachian tube. The method may also comprise providing a retention mechanism configured to retain the tube within the Eustachian tube. In certain embodiments, the retention mechanism may be proximal to the second end of the tube. In certain embodiments, the retention mechanism may be selected from the group consisting of a rib, a flare, a flange, a wedge, a barb, a collar, a hook, and a protrusion. In certain embodiments, the retention mechanism may create a circumferential pressure between the tube and the Eustachian tube during use.

Exemplary embodiments may also comprise providing an endoscope and using the endoscope to guide the tube through the nasopharynx. Exemplary embodiments may also comprise inserting a portion of the endoscope within the aperture, wherein the tube is positioned as a sheath around the endoscope. In certain embodiments, the tube may be comprised of a bioabsorbable or biodegradable material. In specific exemplary embodiments, the tube may be between approximately 5 millimeters and approximately 3 centimeters in length. In certain exemplary embodiments, the diameter of the aperture may be between approximately 1 millimeter and 1 approximately centimeter. Certain embodiments may also comprise inserting at least a portion of the tube into a section of the Eustachian tube that is proximal to the nasopharynx.

Certain exemplary embodiments may also comprise an apparatus comprising: a first end; a second end; and an aperture extending through the apparatus, wherein the apparatus may be configured for insertion through a nasopharynx and into a Eustachian tube. In certain embodiments, the apparatus comprises a retention mechanism. In specific exemplary embodiments, the retention mechanism may be proximal to the second end. In certain exemplary embodiments, the retention mechanism is selected from the group consisting of a rib, a flare, a flange, a wedge, a barb, a collar, a hook, and a protrusion. In specific exemplary embodiments, the retention mechanism creates a circumferential pressure between the apparatus and the Eustachian tube during use.

In exemplary embodiments, the apparatus may comprise a bioabsorbable and/or a biodegradable material. The apparatus may be between approximately 5 millimeters and approximately 3 centimeters in length, and between approximately 1 millimeter and approximately 1 centimeter in diameter.

Exemplary embodiments may also include a kit comprising: a plurality of tubes configured for placement within a Eustachian tube, wherein each of the plurality of tubes comprises: a first end; a second end; and an aperture extending through the tube. In certain embodiments, the kit may comprise a first tube with a first length and a first diameter; a second tube with a second length and a second diameter; and the first length may be greater than the first diameter. In certain embodiments, the first diameter may be greater than the second diameter, while in other embodiments, the first diameter may be less than the second diameter.

While an exemplary embodiment is described herein, it will be understood that various modifications to the method and apparatus can be made without departing from the scope of the present invention. For example, a different configuration for the retention mechanism may be employed and/or the retention may be located in a different position (e.g. proximal to the opposite end of the tube, or approximately equidistant between ends of the tube). In addition, materials of construction may be altered from those mentioned in the exemplary embodiments. The aperture may also be configured differently. For example, the aperture may not extend in a straight path from the first end to the second end. The aperture may enter or exit out the side portions of the tube. Furthermore, the sequential recitation of steps in any claim is not a requirement that the steps be performed in any particular order, unless otherwise so stated.

### BRIEF DESCRIPTION OF THE DRAWINGS

FIG. 1 illustrates a perspective view of an exemplary embodiment of an apparatus according to the present disclosure.

FIG. 2 illustrates a side view of the exemplary embodiment of FIG. 1.

FIG. 3 illustrates a perspective view of an exemplary embodiment of an apparatus according to the present disclosure.

FIG. 4 illustrates a side view of the exemplary embodiment of FIG. 3.

FIG. 5 illustrates a perspective view of an exemplary embodiment of an apparatus according to the present disclosure.

FIG. 6 illustrates a side view of the exemplary embodiment of FIG. 5.

FIG. 7 illustrates a perspective view of an exemplary embodiment of an apparatus according to the present disclosure.

FIG. 8 illustrates a side view of the exemplary embodiment of FIG. 7.

FIG. 9 illustrates a perspective view of an exemplary embodiment of an apparatus according to the present disclosure.

FIG. 10 illustrates a side view of the exemplary embodiment of FIG. 9.

FIG. 11 illustrates a perspective view of an exemplary embodiment of an apparatus according to the present disclosure.

FIG. 12 illustrates a side view of the exemplary embodiment of FIG. 11.

### DETAILED DESCRIPTION OF EXEMPLARY EMBODIMENTS

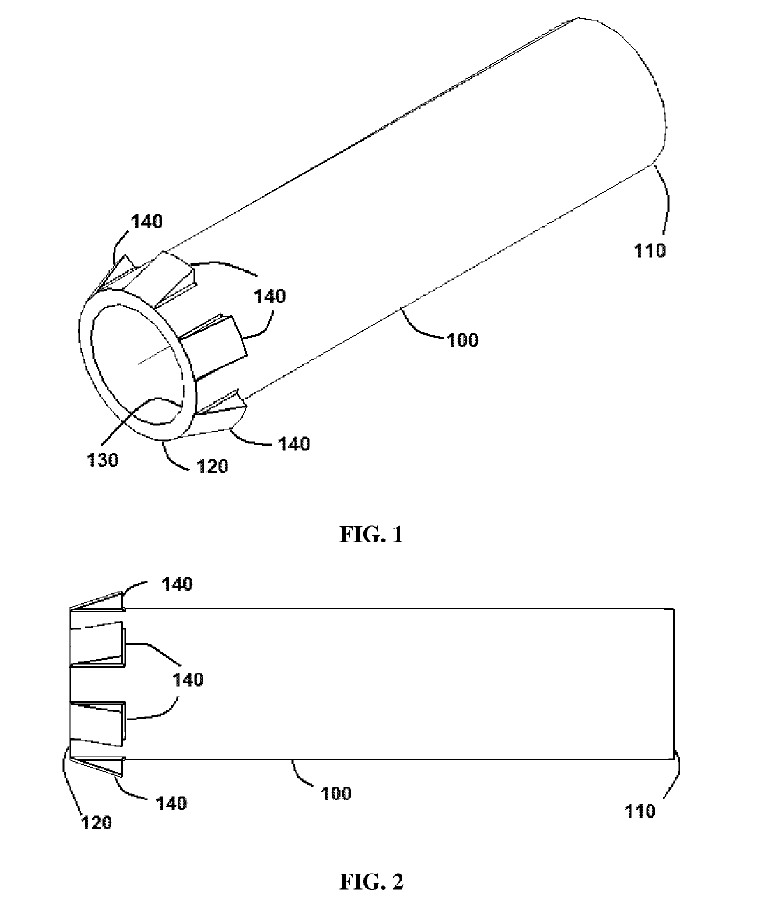

Referring initially to the exemplary embodiment shown in FIGS. 1 and 2, an apparatus for treating Eustachian tube dysfunction comprises a tube 100 having a first end 110, a second end 120, and an internal bore or aperture 130. Tube 100 also comprises a retention mechanism 140 proximal to second end 120. In the exemplary embodiment shown, retention mechanism 140 is configured as a plurality of wedges or barbs distributed around the outer circumference of tube 100.

Tube 100 can be used to provide treatment of Eustachian tube dysfunction (ETD) by inserting tube 100 into a Eustachian tube (not shown) that is or may become obstructed for reasons previously described in this disclosure. Tube 100 can be used to treat ETD by providing a passageway through a Eustachian tube that would otherwise be obstructed. In exemplary embodiments, tube 100 is inserted through the nasopharynx and into the Eustachian tube. In certain exemplary embodiments, an endoscope may be used to assist in guiding tube 100 through the nasopharynx and inserting tube 100 into the Eustachian tube. Tube 100 can be positioned so that second end 120 is inserted into the Eustachian tube. In such embodiments, retention mechanism 140 can be used to assist in securing tube 100 in the Eustachian tube. Tube 100 may be completely inserted into a Eustachian tube in certain embodiments; in other embodiments, only a portion of tube 100 may be inserted.

In certain embodiments, tube 100 comprises a material that is bioabsorbable or biodegradable. In certain exemplary embodiments, the length of tube 100 (i.e., the distance from first end 110 to second end 120) is between approximately 5 millimeters and 3 centimeters. In certain exemplary embodiments, the diameter of aperture 130 is between approximately 1 millimeter and 1 centimeter.

In exemplary embodiments, tube 100 shall remain in the Eustachian tube until it is no longer needed (e.g., the cause of the Eustachian tube obstruction has been eliminated). This timeframe will vary considerably (depending on factors such as the severity of ETD and the patient's response to treatment). In certain embodiments, tube 100 may remain in place for a relatively short period of time, such as a few weeks or months. In other embodiments, the apparatus may remain in place for more than a year. In certain embodiments, tube 100 may comprise a bioabsorbable material that will remain for a certain period of time before degrading (for example, between one and six months, or between two and five months, or between three and four months). In other embodiments, tube 100 may comprise a biocompatible material that can be removed after the underlying cause is effectively corrected.

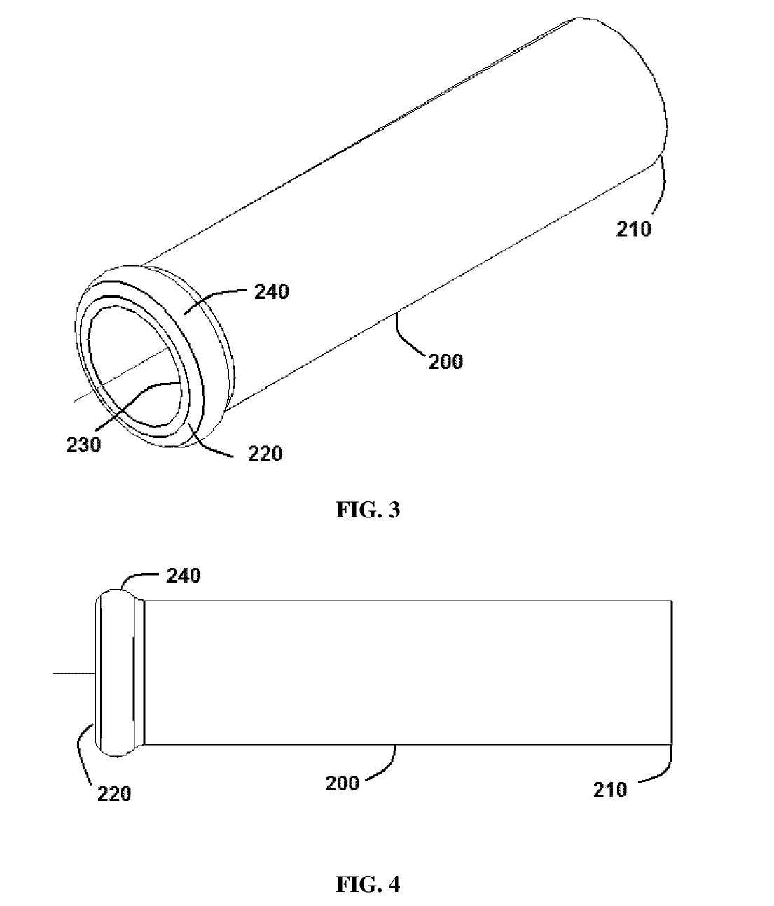

Referring now to FIGS. 3 and 4, an exemplary embodiment comprises a tube 200 comprising a first end 210, a second 220, an internal bore 230 and a retention mechanism 240 proximal to second end 220. In this embodiment, retention mechanism 240 is configured as a collar or protrusion extending around tube 200. Tube 200 is generally equivalent to tube 100 previously described, with the exception of the configuration of retention mechanism 240. Tube 200 can also be used to treat ETD in a manner similar to that described using tube 100.

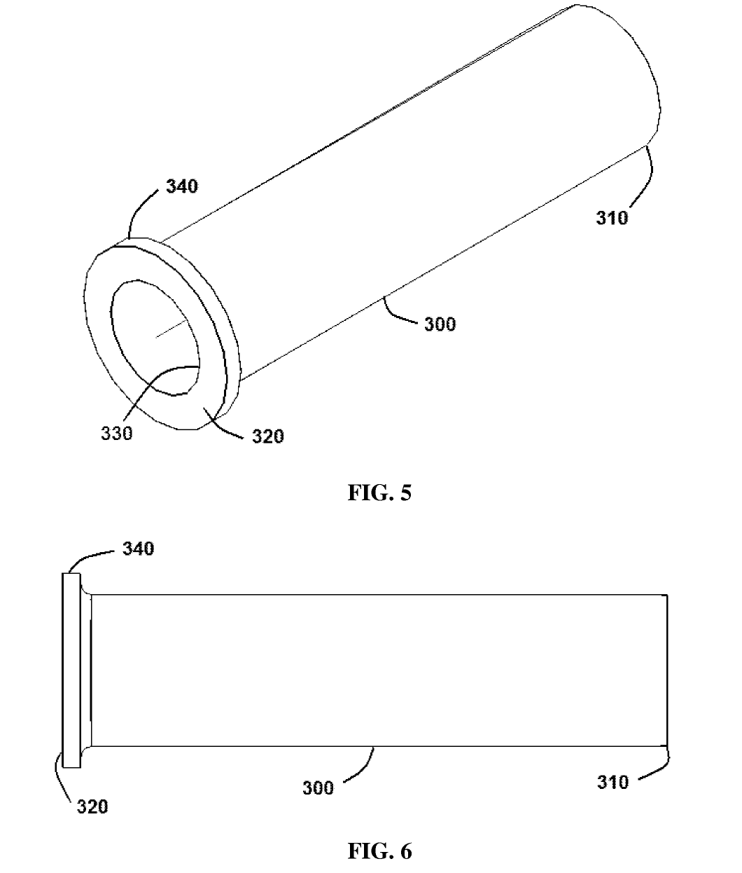

Referring now to FIGS. 5 and 6, an exemplary embodiment comprises a tube 300 comprising a first end 310, a second 320, an internal bore 330 and a retention mechanism 340 proximal to second end 320. In this embodiment, retention mechanism 340 is configured as a flange extending around tube 300. Tube 300 is generally equivalent to tube 100 previously described, with the exception of the configuration of retention mechanism 340. Tube 300 can also be used to treat ETD in a manner similar to that described using tube 100.

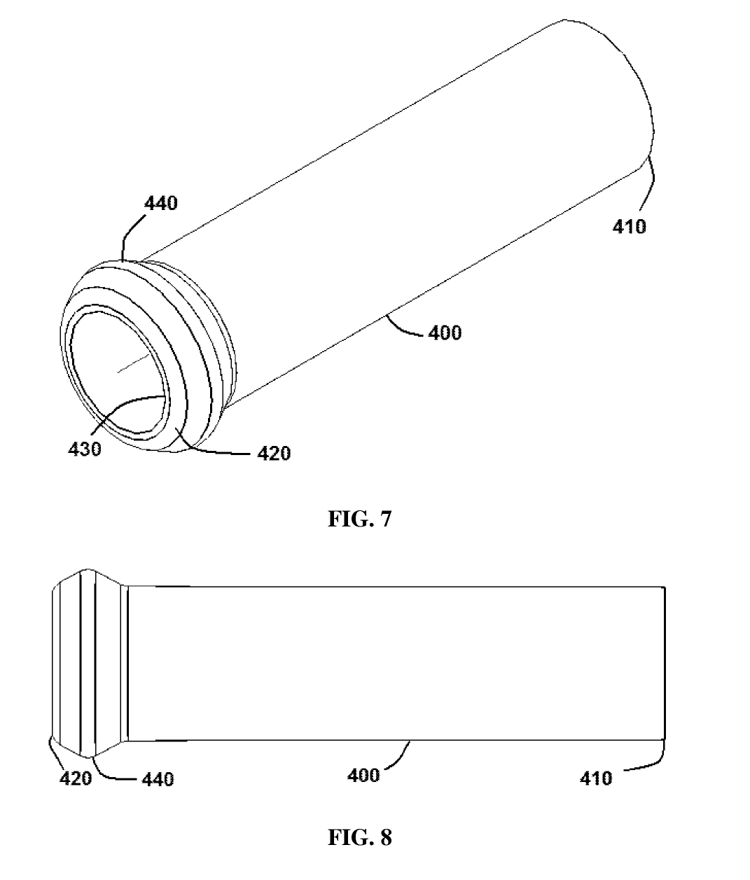

Referring now to FIGS. 7 and 8, an exemplary embodiment comprises a tube 400 comprising a first end 410, a second 420, an internal bore 430 and a retention mechanism 440 proximal to second end 420. In this embodiment, retention mechanism 440 is configured as a rib extending around tube 400. Tube 400 is generally equivalent to tube 100 previously described, with the exception of the configuration of retention mechanism 440. Tube 400 can also be used to treat ETD in a manner similar to that described using tube 100.

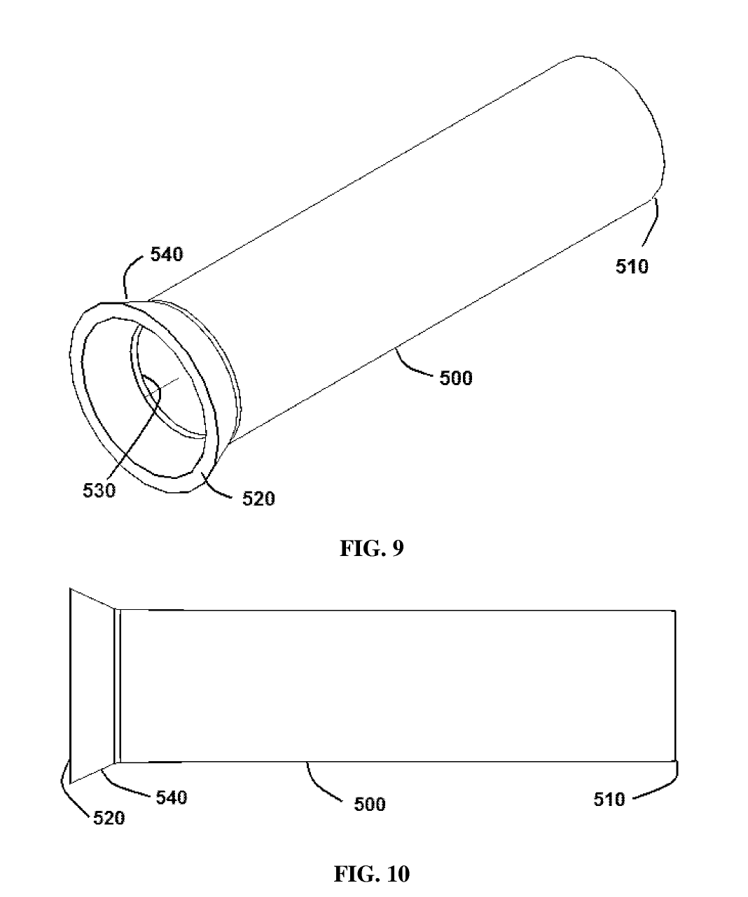

Referring now to FIGS. 9 and 10, an exemplary embodiment comprises a tube 500 comprising a first end 510, a second 520, an internal bore 530 and a retention mechanism 540 proximal to second end 520. In this embodiment, retention mechanism 540 is configured as a flared or tapered portion extending around tube 500. In this embodiment, retention mechanism 540 increases in diameter towards second end 220. Tube 500 is generally equivalent to tube 100 previously described, with the exception of the configuration of retention mechanism 540. Tube 500 can also be used to treat ETD in a manner similar to that described using tube 100.

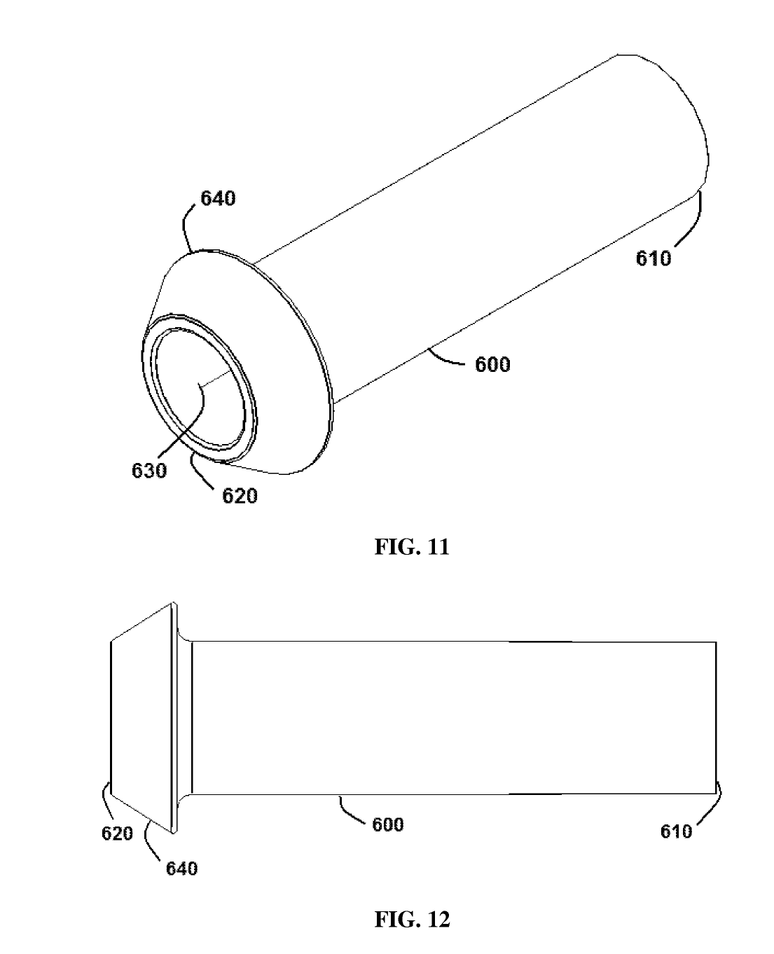

Referring now to FIGS. 11 and 12, an exemplary embodiment comprises a tube 600 comprising a first end 610, a second 620, an internal bore 630 and a retention mechanism 640 proximal to second end 620. In this embodiment, retention mechanism 640 is configured as a flared or tapered portion extending around tube 600. In this embodiment, retention mechanism 640 decreases in diameter towards second end 620. Tube 600 is generally equivalent to tube 100 previously described, with the exception of the configuration of retention mechanism 540. Tube 600 can also be used to treat ETD in a manner similar to that described using tube 100.

1. A method of treating Eustachian tube dysfunction, the method comprising:
providing a tube comprising a first end, a second end, and an aperture extending through the tube;
guiding the tube through a nasopharynx;
inserting at least a portion of the tube into a Eustachian tube; and
retaining at least a portion of the tube within the Eustachian tube.

2. The method of claim 1, father comprising:
providing a retention mechanism configured to retain the tube within the Eustachian tube.

3. The method of claim 2, wherein the retention mechanism is proximal to the second end of the tube.

4. The method of claim 2, wherein the retention mechanism is selected from the group consisting of a rib, a flare, a flange, a wedge, a barb, a collar, a hook, and a protrusion.

5. The method of claim 2, wherein the retention mechanism creates a circumferential pressure between the tube and the Eustachian tube during use.

6. The method of claim 1, further comprising:
providing an endoscope; and
using the endoscope to guide the tube through the nasopharynx.

7. The method of claim 6, further comprising:
inserting a portion of the endoscope within the aperture, wherein the tube is positioned as a sheath around the endoscope.

8. The method of claim 1, wherein the tube comprises a bioabsorbable material.

9. The method of claim 1, wherein the tube comprises a biodegradable material.

10. The method of claim 1, wherein the tube comprises a polymer.

11. The method of claim 1, wherein the tube is between approximately 5 millimeters and approximately 3 centimeters in length.

12. The method of claim 1, wherein the diameter of the aperture is between approximately 1 millimeter and 1 approximately centimeter.

13. The method of claim 1, wherein inserting at least a portion of the tube into a Eustachian tube comprises inserting a portion of the tube into a section of the Eustachian tube that is proximal to the nasopharynx.

14. An apparatus comprising:
a first end;
a second end; and
an aperture extending through the apparatus, wherein:
the apparatus is configured for insertion through a nasopharynx and into a Eustachian tube.

15. The apparatus of claim 14, further comprising:
a retention mechanism.

16. The apparatus of claim 15, wherein the retention mechanism is proximal to the second end.

17. The apparatus of claim 15, wherein the retention mechanism is selected from the group consisting of a rib, a flare, a flange, a wedge, a barb, a collar, a hook, and a protrusion.

18. The apparatus of claim 15, wherein the retention mechanism creates a circumferential pressure between the apparatus and the Eustachian tube during use

19. The apparatus of claim 14 wherein the apparatus comprises a bioabsorbable material.

20. The apparatus of claim 14 wherein the apparatus comprises a biodegradable material.

21. The apparatus of claim 14 wherein the apparatus comprises a polymer.

22. The apparatus of claim 14, wherein the apparatus is between approximately 5 millimeters and approximately 3 centimeters in length.

23. The apparatus of claim 14, wherein the diameter of the aperture is between approximately 1 millimeter and approximately 1 centimeter.

24. A kit comprising:
a plurality of tubes configured for placement within a Eustachian tube, wherein each of the plurality of tubes comprises:
a first end;
a second end; and
an aperture extending through the tube.

25. The kit of claim 24, wherein:
a first tube is a first length and a first diameter;
a second tube is a second length and a second diameter;
and the first length is greater than the first diameter.

26. The kit of claim 25 wherein the first diameter is greater than the second diameter.

27. The kit of claim 26 wherein the first diameter is less than the second diameter.
